(19) United States
(12) Patent Application Publication
(10) Pub. No.: US 2013/0078636 A1
(43) Pub. Date: Mar. 28, 2013

(54) METHOD FOR DETECTING AND QUANTITATING MULTIPLE SUBCELLULAR COMPONENTS

(71) Applicants: Michael Kilpatrick, West Hartford, CT (US); Triantafyllos P. Tafas, Rocky Hill, CT (US)

(72) Inventors: Michael Kilpatrick, West Hartford, CT (US); Triantafyllos P. Tafas, Rocky Hill, CT (US)

(73) Assignee: Ikonisys, Inc., New Haven, CT (US)

(21) Appl. No.: 13/677,884

(22) Filed: Nov. 15, 2012

Related U.S. Application Data
(63) Continuation of application No. 13/047,002, filed on Mar. 14, 2011.
Application No. 13/047,002 is a continuation of application No. 12/108,331, filed on Apr. 23, 2008, now abandoned.
Application No. 12/108,331 is a continuation of application No. 11/924,941, filed on Oct. 26, 2007, now abandoned.
Application No. 11/924,941 is a continuation of application No. 11/233,200, filed on Sep. 22, 2005, now abandoned.
Application No. 11/233,200 is a continuation-in-part of application No. 10/130,559, filed on May 17, 2002, now Pat. No. 7,346,200.
(60) Provisional application No. 60/612,067, filed on Sep. 22, 2004.

(51) Int. Cl. C12Q 1/68

(52) U.S. Cl. CPC C12Q 1/6816
USPC 435/6.11

(57) ABSTRACT

A method for detecting and quantitating multiple and unique fluorescent signals from a cell sample is provided. The method combines immunohistochemistry and a fluorescent-labeled in situ hybridization techniques. The method is useful for identifying specific subcellular components of cells such as chromosomes and proteins.

### CROSS REFERENCE TO RELATED APPLICATIONS

This application is a continuation of pending U.S. patent application Ser. No. 13/047002, filed on Mar. 14, 2011, which is a continuation of U.S. patent application Ser. No. 12/108,331, filed Apr. 23, 2008, which is a continuation of U.S. patent application Ser. No. 11/924,941, filed Oct. 26, 2007, which is a continuation of U.S. patent application Ser. No. 11/233,200, filed Sep. 22, 2005, which is a continuation-in-part application of U.S. patent application Ser. No. 10/130,559, filed on May 17, 2002 (U.S. Pat. No. 7,346,200), which is a national phase application of PCT/US99/27608 (WO 01/37192), filed on Nov. 18, 1999, and claims benefit of U.S. Provisional Patent Application Ser. No. 60/612,067, filed Sep. 22, 2004. The disclosures of which are incorporated by reference herein in their entirety where appropriate for teachings of additional or alternative details, features, and/or technical background.

### BACKGROUND OF THE INVENTION

The invention relates to a method for detecting and quantitating multiple subcellular components of cells using immunostaining and fluorescence-labeled in situ hybridization techniques. In particular, the combination of immunostaining with in situ hybridization allows for the detection of subcellular components in cells, such as fetal hemoglobin in maternal blood samples. The method is useful in prenatal and/or pre-implantation diagnosis of genetic diseases.

A number of techniques exist for the staining and analysis of cells and their components. The ability to simultaneously apply a number of such techniques is highly advantageous for the detailed investigation of specimens in diagnosis of genetic disease has been of special interest. However, combination of prior art techniques have not given any advantages over the single techniques applied alone. Of particular interest, for example, the ability to simultaneously apply immunostaining and fluorescent in situ hybridization (FISH) analysis to a biological specimen offers the potential to obtain quantitative data on, for example, specific protein and nucleic acid components of the same cell at the same time. However, traditional or standard immunostaining and FISH protocols are mutually exclusive. The harsh conditions required for successful FISH analysis are not generally compatible with the retention of significant recognizable antigen, or with the persistence of stable antibody based signal for proper detection of the cellular component. Therefore, there is a need to develop better techniques in the diagnosis of genetic disease using genetic targeting with visualization and quantitation techniques.

### SUMMARY OF THE INVENTION

A single continuous method for the preparation of a biological sample for immunostaining and in situ hybridization analysis is provided.

In one embodiment, a method for identifying multiple cellular components in a cell is provided which method comprises:
reacting a cell sample with at least one antibody, wherein each antibody binds to a specific cellular component and generates a unique fluorescent signal;
treating said cell sample by in situ hybridization using one or more nucleic acid probes; wherein each nucleic acid probe is constructed to hybridize with a target nucleic acid sequence in said cell and generates a unique fluorescent signal;
generating one or more images of said reacted and treated cell sample; and
detecting and analyzing in said image(s) fluorescent signals corresponding to both said antibody and said nucleic acid probe.

### BRIEF DESCRIPTION OF THE DRAWINGS

In the accompanying drawings, in which like reference designations indicate like elements:

FIG. 1 is a flow chart summarizing the method of one embodiment of the invention;

FIG. 2 is a block diagram of an analysis system used in one embodiment of one aspect of the invention;

FIG. 3 is a flow chart of stage I leading to detecting the first signal;

FIGS. 4A and 4B taken together are a flow chart of stage II leading to detecting the first signal;

FIG. 5 is a flow chart of detection of the second signal;

FIG. 6 is a schematic representation of a variation of an apparatus illustrating one embodiment of the invention, using a continuous smear technique;

FIG. 7 is a block diagram of an analysis and reagent dispensing system used in one embodiment of one aspect of the invention;.

FIG. 8 is showing an outline of one embodiment of the invention wherein a multiple objective microscopy system;

FIG. 9 is an image “composition” method;

FIG. 10 is a flowchart of the calibration steps of one embodiment of the invention;

FIG. 11 is a flowchart of the preprocessing steps of one embodiment of the invention; and

FIGS. 12A and 12B are a flowchart of the main processing steps of one embodiment of the invention.

FIG. 13 is a photomicrograph of a combined immunostaining and FISH analysis of cells prepared with the method of the invention as described in Example 1 to identify fetal hemoglobin by immunostaining and the X and Y chromosomes using FISH in the cells.

### DETAILED DESCRIPTION OF THE INVENTION

In embodiments illustrated herein, there is provided a method for detecting and quantitating subcellular components of cells in a cell sample. The method can be applied to a variety of biological samples containing cells, for example, a blood sample, and in particular for the diagnosis of genetic disease in maternal blood.

In one embodiment, the method comprises producing a fluorescent signal generated from one or more antibodies from immunostaining which signals are unique to each antibody used and persist following subsequent treatment of the cell sample for fluorescent in situ hybridization (FISH) analysis. In one embodiment, the methods comprises selecting a desired or unique fluorophore for the FISH probe utilized, which allows discrete visualization and quantitation of each and all fluorescent signals produced, both immunohistochemical and FISH signals fluorescent from the cell sample.

In one embodiment, a method of operating a computer system to detect whether a genetic condition defined by at least one target nucleic acid is present in a sample. The method involves the use of probes and digitized images of the probes hybridized to a sample, together with counting objects and analysis of a statistical expectation to detect whether the genetic condition is present. The counting may involve, for example, counting the number of times a genetic abnormality is detected and comparing that count to a statistical expectation of the abnormality in a particular tissue type, cell type or sample. The counting may involve counting the number of times a genetic abnormality occurs and comparing that count to the number of times a cell type occurs in the same sample or to the number of times a normal nucleic acid occurs in the same sample. The counting may involve counting the number of times more than one different genetic abnormality occurs in a single cell. The computer system also may be used to identify cell type, count cells, examine cell morphology, etc. and compare or correlate this information with the count of the genetic abnormality. Various diagnostic analysis can be carried out.

In one embodiment, it is provided a method of operating a computer system to detect whether a genetic condition defined by at least one target nucleic acid is present in a fixed sample, the method comprising: receiving a digitized image, preferably a color image, of the fixed sample, which has been subjected to fluorescence in situ hybridization under conditions to specifically hybridize a fluorophor-labeled probe to a target nucleic acid and fluorescent immunostaining to detect first objects of interest; processing the image in a computer to separate first objects, for example, a cell component; determining first objects of interest displaying probe associated with the target nucleic acid within specific predetermined characteristics; counting the first objects of interest having probe signals; and analyzing the count of the first objects, for example cells, with respect to a statistical expectation to detect whether the genetic condition is present. This method is applicable to many genetic conditions, including wherein the genetic condition is human trisomy 21. In addition to the foregoing, it will be understood that the statistical expectation can be based on a tissue type, for example. The computer can be used to identify the tissue type of a cell being examined, but the tissue type also can be known.

In some embodiments, the step of receiving further includes a step of producing an image file of red, green and blue pixels representative of red, green and blue intensities at respective pixel locations within the color image received. In some embodiments, the step of processing further includes steps of manually selecting a plurality of pixels within the background; determining color intensity value ranges corresponding to the portion of the background; and identifying as the background those areas of the image having color intensity values within the ranges determined. In some embodiments, before the step of measuring, there may be processing in the computer to filter the color image to make color intensity values of dark pixels in the color image lighter and to make color intensity values of light pixels in the color image darker. The step of filtering may further comprise passing the color image through a hole filling filter; passing the filled color image through an erosion filter; performing a separate operation on the eroded filled color image, to define outlines around areas; selecting pixels within the outlines by performing a logical NOT operation; and performing a logical AND operation between the selected pixels and the filled color image.

In some embodiments, the genetic condition is further defined by a ratio of the target nucleic acid to a second nucleic acid. Then, the method further includes identifying second objects having specific predetermined characteristics associated with the second nucleic acid; and counting second objects identified; wherein analyzing the count of first objects includes finding a ratio of the count of first objects to the count of second objects. In some embodiments, the target nucleic acid defines a dominant trait and the second nucleic acid defines a corresponding recessive trait. The method in those embodiments may include indicating the genetic condition as possessing the dominant trait, possessing the recessive trait, or possessing the dominant trait and carrying the recessive trait depending on the ratio found. When the target nucleic acid is a rearrangement of the second nucleic acid, the method may further include selecting the probe to hybridize with a break region between rearranged and non-rearranged nucleic acids. Finally, the method may include indicating the genetic condition as a severity level related to the ratio found.

According to one embodiment of the invention, there is provided a computer software product comprising: a computer readable storage medium having fixed therein a sequence of computer instructions directing a computer system to count occurrences of a target substance in a cell-containing sample which has been labeled with a target-specific fluorophor, the instructions directing steps of : receiving a digitized color image of the fluorophor-labeled sample; obtaining a color image of the fluorophor-labeled sample; separating objects of interest from background in the color image; measuring parameters of the objects of interest so as to enumerate object having specific characteristics; and analyzing the enumeration of objects with respect to a statistically expected enumeration to determine the genetic abnormality. The instructions can be made to implement all of the variations on the methods described above.

According to another embodiment of the invention, there is provided an apparatus for analyzing an image of a cell-containing sample which has been labeled with a target-specific fluorophor, comprising: a computer system on which image processing software executes; and a storage medium in which is fixed a sequence of image processing instructions including receiving a digitized color image of the fluorophor-labeled sample, obtaining a color image of the fluorophor-labeled sample, separating objects of interest from background in the color image, measuring parameters of the objects of interest so as to enumerate object having specific characteristics, and analyzing the enumeration of objects with respect to a statistically expected enumeration to determine the genetic abnormality. Again, the instructions can be varied to implement all the variations described above.

In yet another embodiment, there is provided a computer-implemented method of processing body fluid or tissue sample image data, the method comprising creating a subset of a first image data set representing an image of a body fluid or tissue sample taken at a first magnification, the subset representing a candidate blob which may contain a rare cell creating a subset of a second image data set representing an image of the candidate blob taken at a second magnification, the subset of the second data set representing the rare cell, storing the subset of the second data set in a computer memory, measuring size and color parameters of the objects of interest so as to identify objects having specific predetermined characteristics associated with the target nucleic acid, counting the objects identified in the step of measuring, and analyzing the count of objects with respect to a statistically expected count to detect whether the genetic abnormality is present.

In one embodiment, there is provided a method including the step of measuring, processing in the computer to filter the color image to make color intensity values of dark pixels in the color image lighter and to make color intensity values of light pixels in the color image darker. Filtering may include the steps of passing the color image through a hole filling filter; passing the filled color image through an erosion filter; performing a separate operation on the eroded filled color image, to define outlines around areas; selecting pixels within the outlines by performing a logical NOT operation, and performing a logical AND operation between the selected pixels and the filled color image.

In one embodiment, a subset of a first image data set can be created by observing an optical field of a monolayer of cells from a body fluid or tissue sample using a computerized microscopic vision system to detect a signal indicative of the presence of a rare cell. In one embodiment, the method can further produce an image file of red, green and blue pixels representative of red, green and blue intensities at respective pixel locations within the color image received. According to some aspects of the invention, the processing further includes manually selecting a plurality of pixels within the background; determining color intensity value ranges corresponding to the portion of the background; and identifying as the background those areas of the image having color intensity values within the ranges determined. In one embodiment, the signal can be measured to determine whether it is a significant signal level. The first and/or the second image data subsets can be transformed into a representation that is more suitable for control and processing by a computer as described herein. the image data is transformed from, for example, a Red Green Blue, (RGB) signal into an Hue Luminescence Saturation (HLS) signal. Filters and/or masks are utilized to distinguish those cells that meet preselected criteria and eliminate those that do not, and thus identify, for example, rare cells.

In another embodiment of the invention, there is provided a method of operating a laboratory service, the method comprising steps of receiving a body fluid or tissue sample, creating a body fluid or tissue sample smear, immunostaining object of interest in the smear with a fluorescent immunostain; treating the smear with a fluorescent probe designed to hybridize with nucleic acid sequences of diagnostic significance; operating a computerized microscope so that a software program automatically identifies objects of interest having hybridized nucleic acid sequences of diagnostic significance based on fluorescent signals generated by the immunostain and nucleic acid probes.

In yet another embodiment of the invention, there is provided computer software product including a computer-readable storage medium having fixed therein a sequence of instructions which when executed by a computer direct performance of steps of detecting objects of interest having nucleic acid sequences of diagnostic significance. The steps encompass: creating a subset of a first image data set representing an image of a body fluid or tissue sample taken at a first magnification, the subset representing a candidate blob which may contain an object of interest, such as a cell or rare cell (less than 1 in 10,000 cells), creating a subset of a second image data set representing an image of the candidate blob taken at a second magnification, the subset of the second data set representing the object of interest, storing the subset of the second data set in a computer memory, measuring fluorescence associated with a fluorescent nucleic acid probe directed to a nucleic acid sequence of diagnostic interest that is associated with objects of interest so as to identify objects having predetermined characteristics associated with the target nucleic acid; counting the objects identified in the step of measuring; and analyzing the count of objects with respect to a statistically expected count to detect whether the genetic abnormality is present.

According to one embodiment of the invention, there is provided a method of preparing a sample of cells for a diagnostic procedure. The sample of cells is obtained and fixed as a monolayer on a substrate, the sample of cells including a rare cell which is present in the sample at no greater than one in every 10,000 cells (i.e. no greater than 0.01%). The monolayer is immunostained with a fluorescent immunostain directed to the rare cell and then treated with a fluorescent probe directed to a nucleic acid sequence associated with a disease sate or abnormality. An optical field covering at least a portion of the sample of cells is observed using a computerized microscopic vision system for fluorescent signals indicative of the presence of a rare cell and the nucleic acid sequence of interest. Each signal is detected, and coordinates where the signals are detected are identified, for the diagnostic procedure. The count of rare cells displaying the nucleic acid sequence associated with a disease state or abnormality may be used to make a diagnosis. A tentative diagnosis may be automatically made by the computerized microscopic system. In one embodiment the rare cell is present at no greater than 0.001% of the cells. In other embodiments the rare cell is present at no greater than 0.0001%, 0.00001% or even 0.000001%.

In another embodiment of the invention, the rare cell type to be detected and diagnosed is a cancer cell found in a sample of cells or tissue from an animal or patient. The sample can be blood or other body fluid containing cells or a tissue biopsy. As an illustration of this embodiment, cancer cell markers described in Section 5, infia, e.g, GM4 protein, telomerase protein or nucleic acids, and p53 proteins or nucleic acids, may be used in the generation of the first or second signal, in a manner to be determined by the specific application of the invention.

In one embodiment of the invention, when the rare cell type is present in the sample, the method of the invention detects the rare cell type at a frequency of no less than 80%. In other embodiments, the detection frequencies are no less than 85%, 90%, 95% and 99%.

According to one embodiment of the invention, there is provided a method of preparing a sample of blood for a diagnostic procedure, which includes: preparing a smear of a sample of unenriched maternal blood containing a naturally present concentration of fetal cells; treating said smear with a fluorescent immunostain directed to said fetal cells; treating said smear with fluorescent nucleic acid probes directed to nucleic acid sequences of interest; observing an optical field covering a portion of the smear using a computerized microscopic vision system for a fluorescent signal indicative of the presence of a fetal cell; and identifying, fetal cells having nucleic acid sequences of interest by way of fluorescent signal from said nucleic acid probes.

In one embodiment, the signal is further processed to represent morphological measurements of the rare cell. In another embodiment, the cells are treated with a label to enhance the optical distinction of rare cells from other cells. In this embodiment, the signal can be, for example, from a label which selectively binds to the rare cells. In another embodiment, the diagnostic procedure involves moving to the coordinates identified and magnifying the optical field until the image is of an isolated rare cell.

In some embodiments, the optical field is stepped over a sequence of portions of the cells covering substantially all of the cells. This may be achieved, for example, by moving the cells on the substrate under control of the computerized microscopic vision system relative to a lens of the computerized microscopic vision system. In another embodiment, the coordinates at which the first signal was obtained are identified, and then the rare cell at those coordinates specifically is contacted after the coordinates have been identified.

In some embodiments, the diagnostic signal can be used to identify the rare cell. In other embodiments, a locating signal can be used to identify the rare cell, and the diagnostic signal is obtained after the cell is located.

In one embodiment, the rare cell is present in the sample at no greater than one in every 10,000 cells (i.e., no greater than 0.01% of the cells). In other embodiments, the rare cell is present at no greater than 0.001%, 0.00001% or even 0.000001%. In one particularly important embodiment, the rare cell is a fetal cell in a sample of cells from maternal blood. Preferably the sample contains only a naturally present concentration of fetal cells which can be no greater than 0.001%, 0.0001%, 0.00001%, 0.000001% or even 0.0000001%.

In any of the foregoing embodiments, the cells can be prepared on, for example a microscope slide or the substrate may have a coordinate system that can be calibrated to the substrate so that coordinates of the rare cell identified in one step can be returned to later in another step. Likewise, the substrate in embodiments has a length that is 10 times its width, the substrate being substantially elongated in one direction. The length can even be 20 times the width. The substrate can be a flexible film, and in one important embodiment, is an elongated flexible film that can carry a relatively large volume of cells, such as would be provided from a relatively large volume of smeared maternal blood. In any of the foregoing embodiments, the fluorescent signal from the immunostain and the fluorescent signal from the nucleic acid probe can be selected whereby they do not mask one another when both are present.

According to embodiments, such methods may employ unenriched or enriched samples, e.g., maternal blood containing naturally present fetal cells.

The invention will be better understood upon reading the following detailed description of the invention and of various exemplary embodiments of the invention, in connection with the accompanying drawings. While the detailed description explains the invention with respect to fetal cells, a rare cell type, and blood as the body fluid or tissue sample, it will be clear to those skilled in the art that the invention can be applied to and, in fact, encompasses diagnosis based on any cell type and any body fluid or tissue sample, particularly where the sample is deposited as a monolayer of cells on a substrate.

Body fluids and tissue samples that fall within the scope of the invention include but are not limited to blood, tissue biopsies, spinal fluid, meningeal fluid, urine, alveolar fluid, etc.. For those tissue samples in which the cells do not naturally exist in a monolayer, the cells can be dissociated by standard techniques known to those skilled in the art. These techniques include but are not limited to trypsin, collagenase or dispase treatment of the tissue.

In one embodiment, the invention is used to detect and diagnose fetal cells. The fluorescent immunostain may be used in an exemplary embodiment to indicate cell identity. For example, the immunostain may be a fluorescent dye bound to an antibody against the hemoglobin ε-chain, i.e., embryonal hemoglobin, for example. Additionally, a metric of each cell's similarity to the characteristic morphology of nucleated erythrocytes, discerned using cell recognition algorithms may be employed to define cell identity.

Diagnosing can be based on the nucleic acid probe signal (or on a combination of a immunostain signal and nucleic probe signal).

In an exemplary embodiment, FISH comprises hybridizing the denatured test DNA of the rare cell type, e. g. a fetal cell, with a denatured dioxygenin (DIG)-labeled genomic probe. The samples containing the test DNA are washed and allowed to bind to an anti-DIG antibody coupled to a fluorophore. Optionally, a second layer of fluorophore (e.g. FITC) is added by incubation with fluorophore-conjugated anti-Fab antibodies. In one embodiment, FISH comprises hybridizing the denatured DNA of the rare cell with a fluorescently labeled probe comprising DNA sequence(s) homologous to a specific target DNA region(s) directly labeled with a particular fluorophore.

Automated sample analysis may be performed by an apparatus and method of distinguishing in an optical field objects of interest from other objects and background. An example of an automated system is disclosed in our U.S. Pat. No. 5,352,613, issued Oct. 4, 1994. Furthermore, once an object has been identified, the color, i.e., the combination of the red, green, blue components for the pixels that comprise the object, or other parameters of interest relative to that object can be measured and stored.

Automated sample analysis and diagnosis of a genetic condition may proceed as follows: (i) receiving a digitized color image of the fixed sample, which has been subjected to fluorescence in situ hybridization under conditions to specifically hybridize a fluorophor-labeled probe to the target nucleic acid; (ii) processing the color image in a computer to separate objects of interest from background in the color image; (iii) measuring parameters of the objects of interest identifying objects having specific characteristics; (iv) counting the objects identified; and (v) analyzing the count of objects with respect to a statistically expected count to determine the genetic condition. The method is useful for diagnosing genetic conditions associated with an aberration in chromosomal number and/or arrangement. Thus, for example, the invention can be used to detect chromosomal rearrangements by using a combination of labeled probes which detect the rearranged chromosome segment and the chromosome into which the segment is translocated. More generally, as well as trisomy, genetic amplifications and rearrangements including translocations, deletions and insertions can be detected using a method embodying this aspect of the invention in connection with properly selected fluorescent probes.

As used herein, “genetic abnormalities” refers to an aberration in the number and/or arrangement of one or more chromosomes with respect to the corresponding number and/or arrangement of chromosomes obtained from a healthy subject, i. e., an individual having a normal chromosome complement. Genetic abnormalities include, for example, chromosomal additions, deletions, amplifications, translocations and rearrangements that are characterized by nucleotide sequences of, typically, as few as about 15 base pairs and as large as an entire chromosome. Genetic abnormalities also include point mutations.

The method is useful for determining one or more genetic abnormalities in a fixed sample, i. e., a sample attached to a solid support which preferably has been treated in a manner to preserve the structural integrity of the cellular and subcellular components contained therein. Methods for fixing a cell containing sample to a solid support, e. g., a glass slide, are well known to those of ordinary skill in the art.

The sample may contain at least one target nucleic acid, the distribution of which is indicative of the genetic abnormality. By “distribution”, it is meant the presence, absence, relative amount and/or relative location of the target nucleic acid in one or more nucleic acids (e. g., chromosomes) known to include the target nucleic acid. In one embodiment, the target nucleic acid is indicative of a trisomy 21 and, thus, the method is useful for diagnosing Down's syndrome. In an embodiment, the sample intended for Down's syndrome analysis is derived from maternal peripheral blood. More particularly, cells are isolated from peripheral blood according to standard procedures, the cells are attached to a solid support according to standard procedures (see, e.g., the Examples) to permit detection of the target nucleic acid.

Fluorescence in situ hybridization refers to a nucleic acid hybridization technique which employs a fluorophor-labeled probe to specifically hybridize to and thereby, facilitate visualization of, a target nucleic acid. Such methods are well known to those of ordinary skill in the art and are disclosed, for example, in U.S. Pat. No. 5,225,326; U.S. patent application Ser. No. 07/668,751; PCT WO 94/02646, the entire contents of which are incorporated herein by reference. In general, in situ hybridization is useful for determining the distribution of a nucleic acid in a nucleic acid-containing sample such as is contained in, for example, tissues at the single cell level. Such techniques have been used for karyotyping applications, as well as for detecting the presence, absence and/or arrangement of specific genes contained in a cell. However, for karyotyping, the cells in the sample typically are allowed to proliferate until metaphase (or interphase) to obtain a “metaphase-spread” prior to attaching the cells to a solid support for performance of the in situ hybridization reaction.

Briefly, fluorescence in situ hybridization involves fixing the sample to a solid support and preserving the structural integrity of the components contained therein by contacting the sample with a medium containing at least a precipitating agent and/or a crosslinking agent. Exemplary agents useful for “fixing” the sample are described in the Examples. Alternative fixatives are well known to those of ordinary skill in the art and are described, for example, in the above-noted patents and/or patent publications.

In situ hybridization may be performed by denaturing the target nucleic acid so that it is capable of hybridizing to a complementary probe contained in a hybridization solution. The fixed sample may be concurrently or sequentially contacted with the denaturant and the hybridization solution. Thus, in one embodiment, the fixed sample is contacted with a hybridization solution which contains the denaturant and at least one oligonucleotide probe. The probe has a nucleotide sequence at least substantially complementary to the nucleotide sequence of the target nucleic acid. The hybridization solution may optionally contains one or more of a hybrid stabilizing agent, a buffering agent and a selective membrane pore-forming agent. Optimization of the hybridization conditions for achieving hybridization of a particular probe to a particular target nucleic acid is well within the level of the person of ordinary skill in the art.

In reference to a probe, the phrase “substantially complementary” refers to an amount of complementarity that is sufficient to achieve the purposes of the invention, i. e., that is sufficient to permit specific hybridization of the probe to the nucleic acid target while not allowing association of the probe to non-target nucleic acid sequences under the hybridization conditions employed for practicing the invention. Such conditions are known to those of ordinary skill in the art of in situ hybridization.

The genetic abnormalities for which the invention is useful include those for which there is an aberration in the number and/or arrangement of one or more chromosomes with respect chromosomes obtained from an individual having a normal chromosome complement. Exemplary chromosomes that may be detected by the present invention include the human X chromosome, the Y chromosome and chromosomes 13, 18 and 21. For example, the target nucleic acid can be an entire chromosome, e.g., chromosome 21, wherein the presence of three copies of the chromosome (“the distribution” of the target nucleic acid) is indicative of the genetic abnormality, Down's syndrome). Exemplary probes that are useful for specifically hybridizing to the target nucleic acid (e. g. chromosome) are probes which can be located to a chromosome(s) that is diagnostic of a genetic abnormality. See e. g., Harrison's Principles of Internal Medicine, 12th edition, ed. Wilson et al., McGraw Hill, N.Y., N.Y. (1991).

One embodiment of the invention is directed to the prenatal diagnosis of Down's syndrome by detecting trisomy 21 (discussed below) in fetal cells present in, for example, maternal peripheral blood, placental tissue, chorionic villi, amniotic fluid and embryonic tissue. However, the method of the invention is not limited to analysis of fetal cells. Thus, for example, cells containing the target nucleic acid may be eukaryotic cells (e. g., human cells, including cells derived from blood, skin, lung, and including normal as well as tumor sources); prokaryotic cells (e. g., bacteria) and plant cells. According to one embodiment, the invention is used to distinguish various strains of viruses. According to this embodiment, the target nucleic acid may be in a non-enveloped virus or an enveloped virus (having a non-enveloped membrane such as a lipid protein membrane). See, e.g., Asgari supra. Exemplary viruses that can be detected by the present invention include a human immunodeficiency virus, hepatitis virus and herpes virus.

The oligonucleotide probe may be labeled with a fluorophor (fluorescent “tag” or “label”) according to standard practice. The fluorophor can be directly attached to the probe (i. e., a covalent bond) or indirectly attached thereto (e.g., biotin can be attached to the probe and the fluorophor can be covalently attached to avidin; the biotin-labeled probe and the fluorophor-labeled avidin can form a complex which can function as the fluorophor-labeled probe in the method of the invention).

Fluorophors that can be used in accordance with the method and apparatus of the invention are well known to those of ordinary skill in the art. These include 4,6-diamidino-2phenylindole (DIPA), fluorescein isothiocyanate (FITC) and rhodamine. See, e. g., the Example. See also U.S. Pat. No. 4,373,932, issued Feb. 15, 1983 to Gribnau et al., the contents of which are incorporated herein by reference, for a list of exemplary fluorophors that can be used in accordance with the methods of the invention. The existence of fluorophors having different excitation and emission spectrums from one another permits the simultaneous visualization of more than one target nucleic acid in a single fixed sample. As discussed below, exemplary pairs of fluorophors can be used to simultaneously visualize two different nucleic acid targets in the same fixed sample.

The distribution of the target nucleic acid is indicative of the genetic abnormality. See e. g., Asgari supra. The genetic abnormalities that may be detected include mutations, deletions, additions, amplifications, translocations and rearrangements. For example, a deletion can be identified by detecting the absence of the fluorescent signal in the optical field. To detect a deletion of a genetic sequence, a population of probes are prepared that are complementary to a target nucleic acid which is present in a normal cell but absent in an abnormal one. If the probe (s) hybridize to the nucleic acid in the fixed sample, the sequence will be detected and the cell will be designated normal with respect to that sequence. However, if the probes fail to hybridize to the fixed sample, the signal will not be detected and the cell will be designated as abnormal with respect to that sequence. Appropriate controls are included in the in situ hybridization reaction in accordance with standard practice known to those of ordinary skill in the art.

A genetic abnormality associated with an addition of a target nucleic acid can be identified, for example, by detecting binding of a fluorophor-labeled probe to a polynucleotide repeat segment of a chromosome (the target nucleic acid). To detect an addition of a genetic sequence (e.g., trisomy 21), a population of probes are prepared that are complementary to the target nucleic acid. Hybridization of the labeled probe to a fixed cell containing three copies of chromosome 21 will be indicated as discussed in the Examples.

Amplifications, mutations, translocations and rearrangements may be identified by selecting a probe which can specifically bind to a break point in the nucleic acid target between a normal sequence and one for which amplification, mutation, translocation or rearrangement is suspected and performing the above-described procedures. In this manner, a fluorescent signal can be attributed to the target nucleic acid which, in turn, can be used to indicate the presence or absence of the genetic abnormality in the sample being tested. The probe may have a sequence that is complementary to the nucleic acid sequence across the break point in a normal individual's DNA, but not in an abnormal individual's DNA. Probes for detecting genetic abnormalities are well known to those of ordinary skill in the art.

An innovative feature of an embodiment of a computer controlled system that may be utilized is an array of two or more objective lenses having the same optical characteristics. The lenses are arranged in a row and each of them has its own z-axis movement mechanism, so that they can be individually focused. This system may be equipped with a suitable mechanism so that the multiple objective holder can be exchanged to suit the same variety of magnification needs that a common single-lens microscope can cover.

Each objective may be connected to its own CCD camera. Each camera may be connected to an image acquisition device. For each optical field acquired, the computer may record its physical location on the microscopical sample. This may be achieved through the use of a computer controlled x-y mechanical stage. The image provided by the camera is digitized and stored in the host computer memory.

The computer may keep track of the features of the objectives-array in use as well as the position of the motorized stage. The stored characteristics of each image can be used in fitting the image in its correct position in a virtual patchwork, i.e. “composed” image, in the computer memory.

The host computer system may be driven by software system that controls all mechanical components of the system through suitable device drivers. The software may comprise image composition algorithms that compose the digitized image in the computer memory and supply the composed image for processing to further algorithms. Through image decomposition, synthesis and image processing specific features particular to the specific sample may be detected.

In one embodiment both the immunostain signals and probe signals are detected simultaneously. The signals may be processed separately (with signals from different fluorophores for the immunostain and probe also being processed separately). In an embodiment, the simultaneous presence of both immunostain and probe signals at a single set of coordinates or even a single signal which results from the interaction of two components (e. g. a quenching of a signal by a partner ‘signal’) may be used for diagnostic purposes.

Generally the materials and techniques used to generate the immunostain signal should not interfere adversely with the materials and techniques used to generate the second probe (to an extent which compromises unacceptably the diagnosis), and visa versa. Nor should immunostain or probe damage or alter the cell characteristics sought to be measured to an extent that compromises unacceptably the diagnosis. Finally, any other desirable or required treatment of the cells should generally not interfere with the materials or techniques used to generate the first and second signals to an extent that compromises unacceptably the diagnosis. Within those limits, any suitable generators of the first and second signals may be used.

In one embodiment of the invention, when a rare cell type is to be detected, the method of the invention detects the rare cell type at a frequency of no less than 80%. In other embodiments, the detection frequencies are no less than 85%, 90%, 95% and 99%.

While the of single fluorophores for the tagging of an individual allele may create an upper limit as to the number of mutations that can be tested simultaneously, the use of combinatorial chemistry may be employed to the number of allele specific mutations that can be tagged and detected simultaneously. Chromosomal abnormalities that fall within the scope of the invention include but are not limited to Trisomy 21,18,13 and sex chromosome aberrations such as XXX, XXY, XYY. With the use of combinatorial chemistry, the methods of the invention can be used to diagnose a multitude of rearrangements, including translocations observed in genetic disorders and cancer. Mendelian disorders that fall within the scope of the invention include but are not limited to cystic fibrosis, hemochromatosis, hyperlipidemias, Marfan syndrome and other heritable disorders of connective tissue, hemoglobinopathies, Tay-Sachs syndrome or any other genetic disorder for which the mutation is known. The use of combinatorial chemistry dyes allows for the simultaneous tagging and detection of multiple alleles thus making it possible to establish the inheritance of predisposition of common disorders, e. g. asthma and/or the presence of several molecular markers specific for cancers, e. g., prostate, breast, colon, lung, leukemias, lymphomas, etc.

One use of the invention is in the field of cancer. Cancer cells of particular types often can be recognized morphologically against the background of noncancer cells. The morphology of cancer cells therefore can be used as the first signal. Heat shock proteins also are markers expressed in most malignant cancers. Labeled antibodies, such as fluorescently-tagged antibodies, specific for heat shock proteins can be used to generate the first signal. Likewise, there are antigens that are specific for particular cancers or for particular tissues, such as Prostate Specific Antigen, and antibodies specific for cancer or tissue antigens, such as Prostate Specific Antigen can be used to generate a first signal for such cancer cells.

Thus, rare cancer cells in a background of other cells can be identified and characterized according to the invention. The characterization may include a confirmation of a diagnosis of the presence of the cancer cell, a determination of the type of cancer, a determination of cancer risk by determining the presence of a marker of a genetic change which relates to cancer risk, etc.

Markers of genetic changes enable assessment of cancer risk. They provide information on exposure to carcinogenic agents. They can detect early changes caused by exposure to carcinogens and identify individuals with a particularly high risk of cancer development. Such markers include LOH on chromosome 9 in bladder cancer, and chromosomelp deletions and chromosome 7,17 and 8 gains/losses detected in colorectal tumorigenesis.

Development of lung cancer requires multiple genetic changes. Activation of oncogenes includes K-ras and myc. Inactivation of tumor suppressor genes includes Rb, p53 and CDKN2. Identification of specific genes undergoing alteration is useful for the early detection of cells destined to become malignant and permits identification of potential targets for drugs and gene-based therapy.

In determining trisomy, the invention contemplates determining the presence of trisomy in a single cell, and/or determining the frequency of single cells with trisomy in a population of cells (which could be done without knowing which cells are trisomic; i. e. total number of cells counted and total number of chromosomes counted). The existence of trisomy or the risk of a condition associated with trisomy then could be evaluated.

Important is the recognition that signals can be counted and be compared to other information (e. g. other signal counts, statistical information about predicted signal frequency for different tissue types, etc.) so as to yield relevant diagnostic information.

The invention also has been described in connection with identifying a pair of signals, one which identifies a target rare cell such as a fetal cell and another which is useful in evaluating the state of the cell such as a fetal cell having a genetic defect. It should be understood that according to certain embodiments, only a single signal need be detected. For example, where a fetal cell carries a Y chromosome and the diagnosis is for an abnormality on the Y chromosome, then the signal which identifies the genetic abnormality can be the same as that which identifies the fetal cell. As another example, a single signal can be employed in circumstances where the observed trait is a recessive trait. A pair of signals also can be used to detect the presence of two alleles or the existence of a condition which is diagnosed by the presence of two or more mutations in different genes. In these circumstances the pair of signals (or even several signals) can identify both the phenotype and the cell having that phenotype. Such embodiments will be apparent to those of ordinary skill in the art.

### EXEMPLARY EMBODIMENTS

### Example 1

The following procedure for analyzing blood samples for the presence of cells containing fetal hemoglobin using an immunostaining technique and to determine the presence of the X and Y chromosomes in the same cells by a fluorescent-labeled in situ hybridization technique.

Cells are deposited on a solid support suitable for microscopic analysis and fixed with methanol. Following air drying, cells are rinsed in phosphate buffered saline and further fixed in 2% formaldehyde in phosphate buffered saline. Cells are then washed sequentially in phosphate buffered saline, followed by Tris-buffered saline, pH 7.6 containing Tween® 20. Following removal of excess liquid, blocking agent is added and the slides incubated in a humidified chamber. After the blocking solution is removed, a dilution of primary antibody in blocking agent is added and the cells incubated for 30 to 120 minutes in a humidified chamber. The antibody solution is then removed and the cells rinsed several times in Tris-buffered saline pH 7.6 containing Tween®20. Excess liquid is removed, and a dilution of anti-mouse secondary antibody in blocking agent is added, and the cells are incubated in a humidified chamber for 30 to 120 minutes. The antibody solution is then removed and the cells again rinsed several times in Tris-buffered saline, pH 7.6 containing Tween® 20. After removal of excess fluid, a fresh, filtered solution of HNPP/Fast Red dye in Alkaline phosphatase buffer is added and the cell sample is incubated for 10 minutes. The staining solution is removed and the cells rinsed in Tris-buffered saline, pH 7.6, containing Tween® 20, followed by a solution of DAPI in Tris-buffered saline pH 7.6 containing Tween® 20. The cells are rinsed twice in Tris-buffered saline, pH 7.6 containing Tween® 20 and then in standard saline citrate, excess liquid removed and the cells are air dried. The cells are then incubated in pre-warmed 0.005% pepsin at 37° C. for 5 minutes. The cells are then washed in 50 mM MgCl₂ in phosphate buffered saline for 5 minutes, then twice in phosphate buffered saline, excess liquid removed and the cells dried. A solution of fluorescently labeled FISH probe, such as DNA and or RNA, in hybridization is then added, a coverslip applied on top of the slide containing the cells, and then cells incubated at 74° C. for 2.5 minutes, then at 37° C. for 4 to 16 hours in a humidified chamber. The coverslip is removed and the cells washed in 0.4× standard saline citrate at room temperature for 2 minutes. Excess liquid is removed and the cells air dried and mounted for microscope observation and analysis.

### Example 2—Apparatus

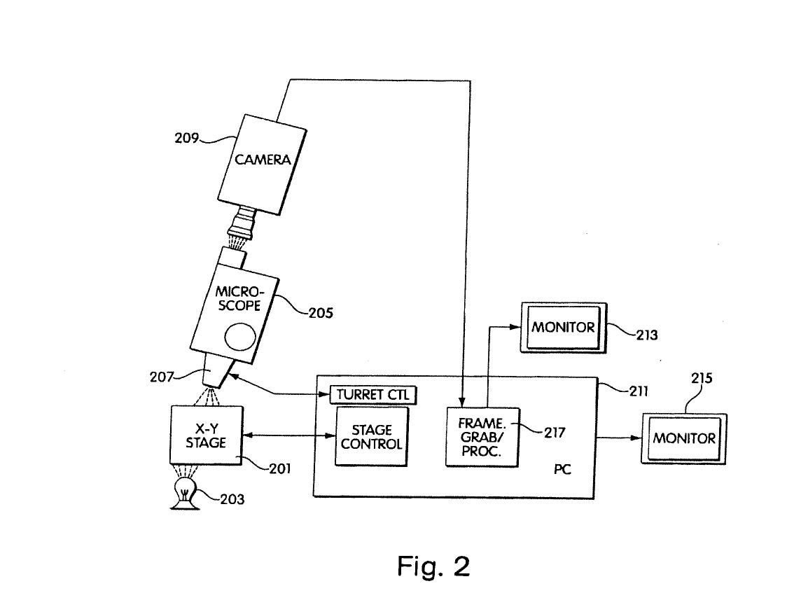

The block diagram of FIG. 2 shows the basic elements of an embodiment system suitable for embodying this aspect of the invention. The basic elements of such system include an X-Y stage 201, a mercury light source 203, a fluorescence microscope 205 equipped with a motorized objective lens turret (nosepiece) 207, a color CCD camera 209, a personal computer (PC) system 211, and one or two monitors 213,215.

The individual elements of the system can be custom built or purchased offthe-shelf as standard components. Each element will now be described in somewhat greater detail.

The X-Y stage 201 can be any motorized positional stage suitable for use with the selected microscope 205. Preferably, the X-Y stage 201 can be a motorized stage that can be connected to a personal computer and electronically controlled using specifically compiled software commands. When using such an electronically controlled X-Y stage 201, a stage controller circuit card plugged into an expansion bus of the PC 211 connects the stage 201 to the PC 211. The stage 201 should also be capable of being driven manually. Electronically controlled stages such as described here are produced by microscope manufacturers, for example including Olympus (Tokyo, Japan), as well as other manufacturers, such as LUDL (NY, USA).

The microscope 205 may be, for example, any fluorescence microscope equipped with a reflected light fluorescence illuminator 203 and a motorized objective lens turret 207 with a 20× and an oil immersion 60× or 63× objective lens, providing a maximum magnification of 600×. The motorized nosepiece 207 is preferably connected to the PC 211 and electronically switched between successive magnifications using specifically compiled software commands. When using such an electronically controlled motorized nosepiece 207, a nosepiece controller circuit card plugged into an expansion bus of the PC 211 connects the stage 201 to the PC 211. The microscope 205 and stage 201 are set up to include a mercury light source 203, capable of providing consistent and substantially even illumination of the complete optical field.

The microscope 205 produces an image viewed by the camera 209. The camera 209 can be any color 3-chip CCD camera or other camera connected to provide an electronic output and providing high sensitivity and resolution. The output of the camera 209 is fed to a frame grabber and image processor circuit board installed in the PC 211. A camera found to be suitable is the SONY 930 (SONY, Japan).

Various frame grabber systems can be used in connection with the present invention. The frame grabber can be, for example a combination of the MATROX IM-CLD (color image capture module) and the MATROX IM-640 (image processing module) set of boards, available from MATROX (Montreal, CANADA). The MATROX IM-640 module features on-board hardware supported image processing capabilities. These capabilities compliment the capabilities of the MATROX IMAGINGLIBRARY (MIL) software package. Thus, it provides extremely fast execution of the MIL based software algorithms. The MATROX boards support display to a dedicated SVGA monitor. The dedicated monitor is provided in addition to the monitor usually used with the PC system 211. Any monitor SVGA monitor suitable for use with the MATROX image processing boards can be used. One dedicated monitor usable in connection with the invention is a ViewSonic 4E (Walnut Creek, Calif.) SVGA monitor. In order to have sufficient processing and storage capabilities available, the PC 211 can be any INTEL PENTIUM-based PC having at least 32 MB RAM and at least 2GB of hard disk drive storage space. The PC 211 preferably further includes a monitor. Other than the specific features described herein, the PC 211 is conventional, and can include keyboard, printer or other desired peripheral devices not shown.

The PC 211 may execute a smear analysis software program compiled in MICROSOFT C++ using the MATROX IMAGING LIBRARY (MIL). MIL is a software library of functions, including those which control the operation of the frame grabber 211 and which process images captured by the frame grabber 211 for subsequent storage in PC 211 as disk files. MIL comprises a number of specialized image processing routines particularly suitable for performing such image processing tasks as filtering, object selection and various measurement functions. The smear analysis software program may run as a WINDOWS 95 application. The program prompts and measurement results are shown on the computer monitor 213, while the images acquired through the imaging hardware 211 are displayed on the dedicated imaging monitor 215.

In order to process microscopic images using the smear analysis program, the system is first calibrated. Calibration compensates for day to day variation in performance as well as variations from one microscope, camera, etc., to another. During this phase a calibration image is viewed and the following calibration parameters are set:
the color response of the system;
the dimensions or bounds of the area on a on a slide containing a smear to be scanned for fetal cells;
the actual dimensions of the optical field when using magnifications 20× and 60× (or 63×); and
the minimum and maximum fetal nuclear area when using magnifications 20× and 60× (or 63×).

### Detection of an Object Identification Signal

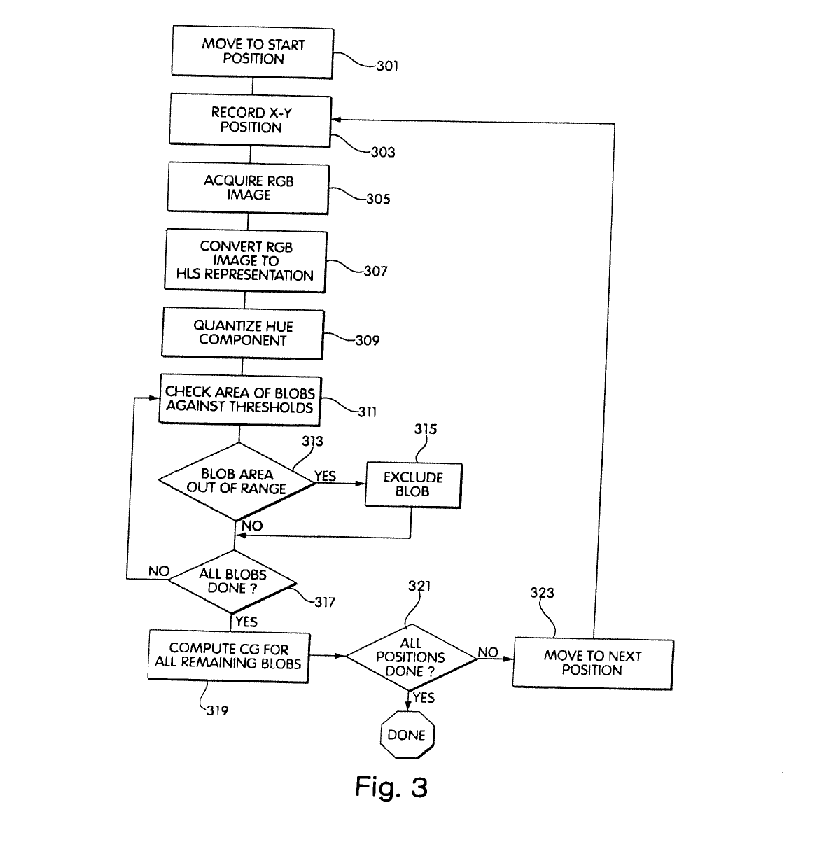

The detection algorithm may operate in two stages. The first may be a prescan stage I, illustrated in embodiment the flow chart of FIG. 3, where possible fetal cell positions are identified using a low magnification and high speed. The 20× objective may be, for example, selected and the search of fetal cells can start:
The program moves the automated stage (FIG. 2,201) to a preset starting point, for example one of the corners of a slide containing a smear (Step 301).
The x-y position of the stage at the preset starting point is recorded (Step 303) optical field.
The optical field is acquired (Step 305) using the CCD camera 209 and transferred to the PC 211 as an RGB (Red/Green/Blue) image.
The RGB image is transformed (Step 307) to the HLC_(Hue/Luminance/Saturation) representation.
The Hue component is binary quantized (Step 309) as a black and white image so that pixels with Hue values ranging between 190 and 255 are set to 0 (black) representing interesting areas (blobs), while every other pixel value is set to 255 (white, background). The blobs represent possible fetal cell nuclear areas.
The area of each blob in the binary quantized image is measured. If, at 20× magnification, it is outside a range of about 20 to 200 pixels in size, the blob's pixels are set to value 255 (background); they are excluded from further processing (Steps 311,313,315 and 317).
Then the coordinates of each blob's center of gravity (CG) are calculated (Step 319), using a custom MATROX function. The center of gravity of a blob is that point at which a cut-out from a thin, uniform density sheet of material of the blob shape would balance. These coordinates are stored in a database along with the z-y position of the current optical field, so the blob can be located again at the next processing stage using higher magnification.
Additional optical fields are processed similarly, recording the x-y position of each succeeding optical field, until the complete slide are is covered (Steps 321 and 323).

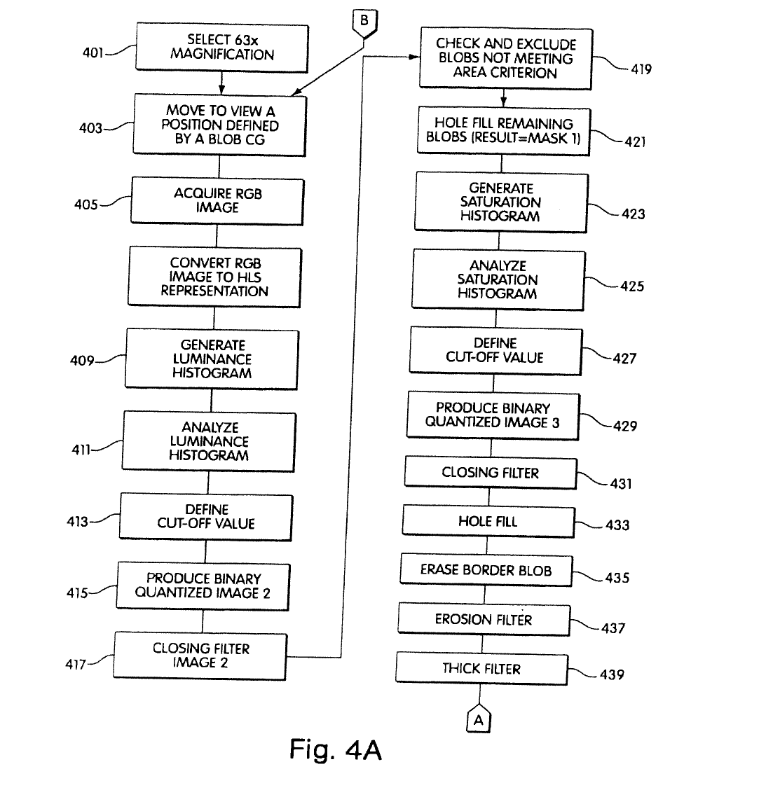

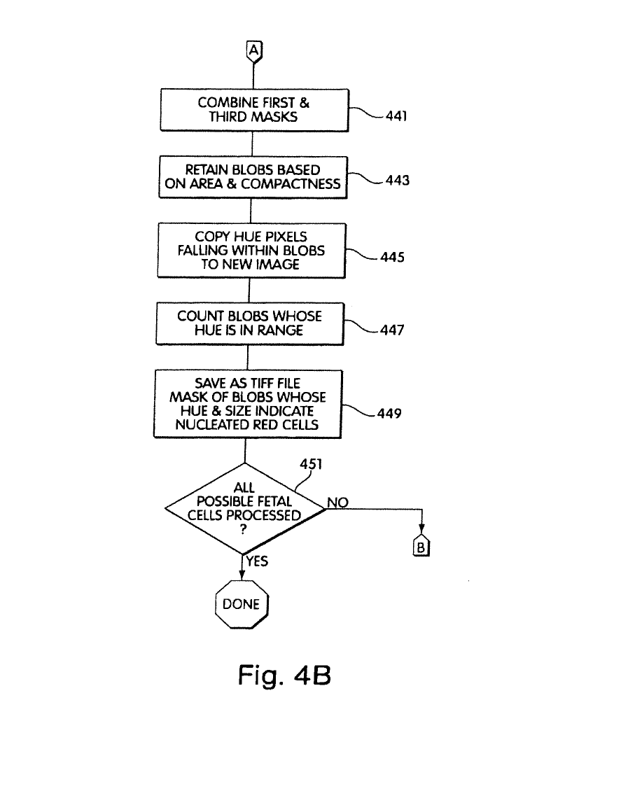

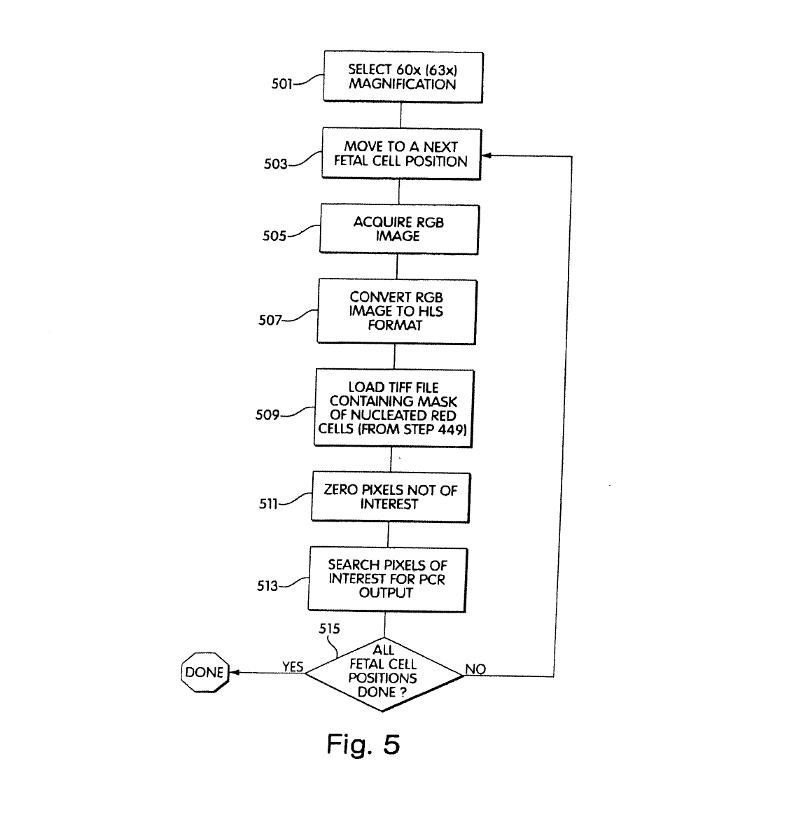

Stage II, illustrated in embodiment flow chart of FIGS. 4 A and 4 B, includes the final fetal cell recognition process:
63× magnification is selected (Step 401).
The program moves the automated stage (FIG. 2,201) so that the coordinates of the first position of a CG found earlier, which is possible fetal cell nuclear area, is at the center of the optical field (Step 403).
The optical field is acquired using the CCD camera (FIG. 2,209) and transferred to the computer as an RGB image (Step 405).
The RGB image is transformed to the HLS model (Step 407).
The program then generates a Luminance histogram (Step 409) by counting the number of pixels whose Luminance value equals each possible value of Luminance. The counts are stored as an array of length 256 containing the count of pixels having a grey-level value corresponding to each index into the array.
The program next analyzes the Luminance distribution curve (Step 411), as represented by the values stored in the array, and locates the last peak. It has been found that this peak includes pixel values that represent plasma area in the image. The function that analyzes the Luminance distribution curve: calculates a 9-point moving average to smooth the curve; calculates the tangents of lines defined by points
   10 grey-level values distant; calculates the slopes of these lines in degrees; finds the successive points where the curve has zero slope and sets these points (grey-levels) as −1 if they represent a minimum (valley in the curve) or 1 if they represent a maximum (peak in the curve); then finds the locations of peaks or valleys in the curve by finding the position of a 1 or −1 in the array of grey-level values.
The program then sets as a cut-off value the grey-level value of pixels lying in the valley of the Luminance distribution which occurs before the last peak of the distribution (Step 413).
Using this cut-off value, the program then produces (Step 415) a second binary quantized image. This is a black-and-white image in which pixels corresponding to pixels in the Luminance image having grey-level values lower than the cut off point are set to 255 (white) and pixels corresponding to pixels in the Luminance image having grey-level values higher than the cut off point are set to 0 (black). The white blobs of this image are treated as cells while the black areas are treated as non-cellular area.
A closing filter is applied (Step 417) to the second binary quantized image; in this way holes, i. e., black dots within white regions, are closed.
The program now measures the area of the cells. If the area of any of the cells is less than 200 pixels then these cells are excluded, i. e. the pixels consisting these cells are set to pixel value 255 (black) (Step 419).
A hole fill function, found in the MIL, is applied to the remaining blobs (Step 421).
The resulting binary quantized image, after processing, is a mask whose white regions denote only cells.
Red blood cells are now distinguished from white blood cells based on the Saturation component of the HLS image. The mask is used to limit processing to only the cell areas.
The program now counts the number of pixels whose Saturation value is each possible value of Saturation. The counts are stored as an array of length 256 containing the count of pixels having a grey-level value corresponding to each index into the array (Step 423).
The program now analyzes (Step 425) the Saturation distribution curve, as represented by the values stored in the array, and locates the first peak. This peak includes pixel values that represent areas contained in white blood cells.
The grey-level value that coincides with the first minimum (valley) after the peak is set as a cut-off point (Step 427).
Using this cut-off value the program produces (Step 429) a third binary quantized image. Pixels corresponding to pixels in the Saturation image having grey-level values higher than the cut-off point are set to 255 (white). They constitute red blood cell areas. Pixels corresponding to pixels in the Saturation image having grey-level values lower than the cutoff point are set to 0 (black). The white blobs of this third binary quantized image are seeds for areas that belong to red blood cells.
A closing filter is applied (Step 431) to the third binary quantized image; in this way holes, i. e., black dots within white regions, are closed.
A hole fill function, found in the MIL, is applied (Step 433) to the remaining blobs.
The resulting binary quantized image, after processing, is a new mask that contains only white blood cells.
An erase border blob function of MIL is now applied (Step 435) to the remaining blobs, removing those which include pixels coincident with a border of the image area. Such blobs cannot be included in further processing as it is not known how much of the cell is missing when it is coincident with a border to the image area.
An erosion filter is applied 6 times to this mask; thus any connected blobs (white blood cell seeds) are disconnected (Step 437).
A “thick” filter is applied 14 times (Step 439). The “thick” filter is equivalent to a dilation filter. That is, it increases the size of a blob by successively adding a row of pixels at the periphery of the blob. If a growing blob meets an adjacent blob growing next to it, the thick filter does not connect the two growing blobs. Thus adjacent blobs can be separated.
The first binary quantized mask (containing all the cells) and the third binary quantized mask (containing the separated seeds of white blood cells) are combined with a RECONSTRUCTFROMSEED MIL operator. A fourth mask thus constructed contains blobs (cells) copied from the first mask that are allowed by the third mask and therefore represent white blood cells (Step 441).
The blobs in the fourth mask are measured for their area and compactness: Area (A) is the number of pixels in a blob; Compactness is derived from the perimeter (p) and area (A) of a blob, it is equal to: p2/4 (A). The more convoluted the shape, the bigger the value. A circle has the minimum compactness value (1.0). Perimeter is the total length of edges in a blob, with an allowance made for the staircase effect which is produced when diagonal edges are digitized (insidecorners are counted as 1.414, rather than 2.0). Blobs are retained in the fourth mask only if their area is between 1000 and 8000 pixels and they have a compactness less than 3, thus allowing for cells with relatively rough outline. Blobs that touch the border of the image are excluded from further processing (Step 443).
The fourth mask is applied to the Hue component in the following manner (Steps 445, 447,449 and 451):
Pixels from the Hue component are copied to a new image retaining their Hue value, provided that their coordinates coincide with white (255) pixels in the “mask”; all other pixels in the new image are set to 0 (black) (Step 445).
The pixel values in each of the contiguous non-0 pixel areas, i. e., those blobs corresponding to images of red cells, are checked for values between 190 and 255.
   The number of such pixels in each blob is counted (Step 447).
If there are more than 200 such pixels, the blob represents a nucleated red blood cell. The coordinates of the center of gravity of each such cell are stored. The mask is binary quantized so that all pixels having non-0 values are set to 255 (white); and the mask is stored as a separate Tagged Image File Format (TIFF) file (Step 449).
The program moves to the next stored coordinates for a possible fetal cell which do not coincide with any of the coordinates stored during the previous step. The entire process is repeated until a preset number of nucleated red blood cells have been identified. The results, including the nucleated red blood cell coordinates and the names of the respective mask files, along with various characteristic codes for the blood slide are stored in a result text file. The nucleated red blood cells whose coordinates are stored are the fetal cells sought (Step 451).

After the object of interest, such as the fetal cells, are identified, the second signal is generated, for example by in situ PCR or PCR in situ hybridization or FISH, as described above.

### Detection of the Diagnostic Signal

A smear including in situ PCR or PCR in situ hybridization treated cells is positioned on the stage (FIG. 2,201). If necessary calibration steps are taken, as before. Calibration permits the software to compensate for day to day variation in performance as well as variations from one microscope, camera, etc. to another. Detection of the diagnostic signal in an embodiment method may proceed as shown in the flow chart of FIG. 4, as follows:
Magnification objective 60× (63×) is chosen (Step 501).
The x-y stage is moved to the first fetal cell position according to data from the result file compiled from detection of the first signal, as described above (Step 503).
The optical field is acquired using the CCD camera (FIG. 2,209) and transferred to the computer (FIG. 2,211) as an RGB image (Step 505).
The RGB image is transformed to the HLS model (Step 507).
The TIFF file containing the black and white mask is loaded as a separate image (Step 509).
The pixels of the Hue component not corresponding to white areas in the mask arc set to 0 (black) (Step 511).
The remaining areas, which represent fetal cells, are searched for pixel values corresponding to a signal produced following PCR. For example, the signal may be a color which arises due to the presence of alkaline phosphatase, i. e., red. The non black areas of the Hue component are searched for pixel values ranging from 0 to 30 (Step 513).
The stage is moved to the next non-processed fetal cell and the above process is repeated (Step 515).

The PC 211 executes a software program called SIMPLE which controls operation of the frame grabber and image processor circuit 217. SIMPLE also processes images captured by frame grabber and image processor circuit 217 and subsequently stores images and processed data in PC 211 as disk files. SIMPLE provides an icon-based environment with specialized routines particularly suitable for performing such image processing tasks as filtering, object selection and measurement. Most of the SIMPLE tasks are directed by a human operator using a pointing device connected to PC 211, such as a mouse or trackball (not shown).

In order to process images using SIMPLE, a number of image calibration steps must first be taken. In an embodiment, a new slide properly stained using the fluorescence in situ hybridization (FISH) technique is placed under the fluorescence microscope. The objects of interest which are to be recognized, i. e., the nuclear or chromosomal areas, have specific chromatic features. Multiple targets can be delineated simultaneously in a particular specimen by combining fluorescence detection procedures. That is, if different targets are labeled with different fluorophors that fluoresce at different wavelengths, then the software program can be made to separately identify objects emitting the different fluorophors, provided full color information is available in the image. Targets with differing affinities for different fluorophors may be differentiated by the color combinations emitted. Each target may emit at wavelengths corresponding to two or more fluorophors, but the intensity of each may differ, for example. Thus, all three color components of the microscopic images are used during processing.

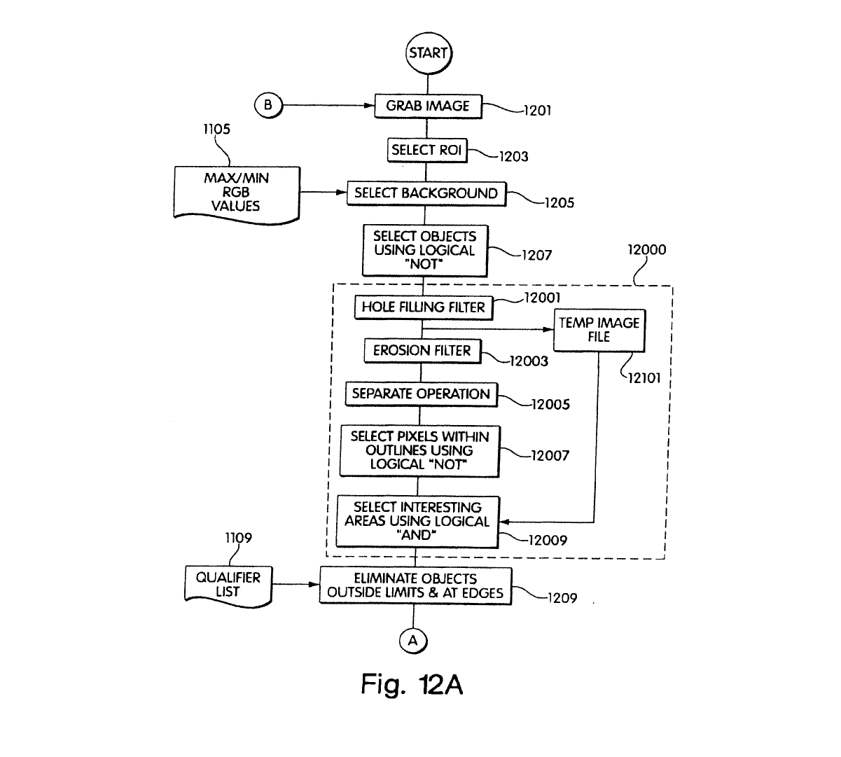

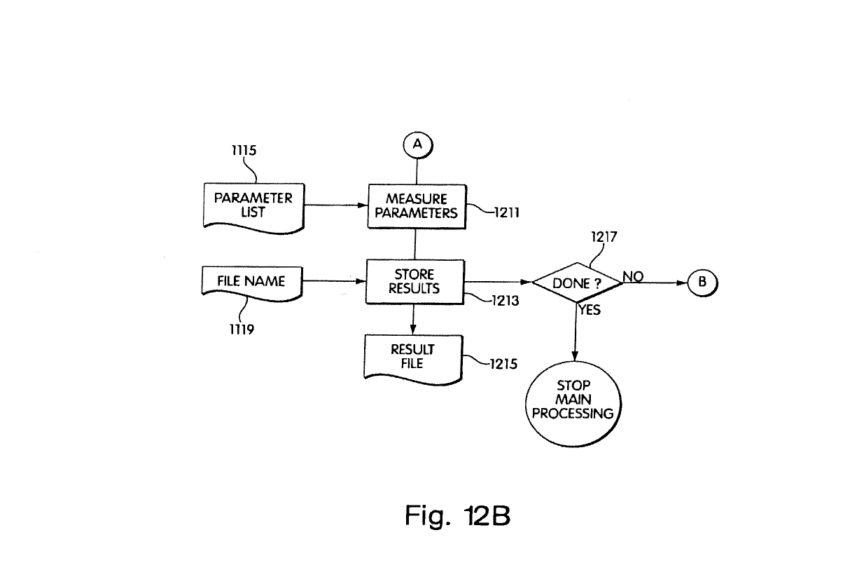

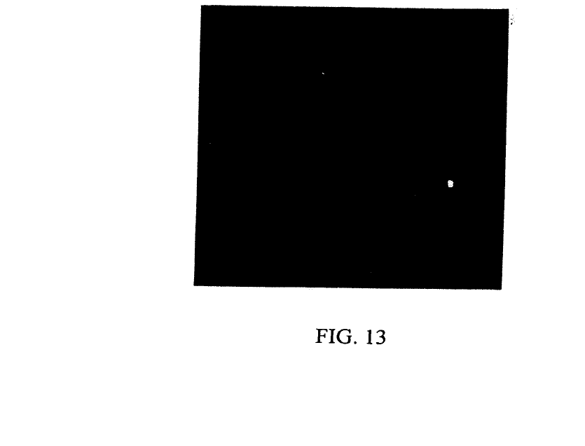

For each new specimen inserted under the microscope, a preprocessing procedure is first executed. The flowchart of FIG. 12A shows the preprocessing steps of this embodiment of the present invention. Preprocessing may be used to permit the software to compensate for specimen-to-specimen variations.

In one embodiment, the slide containing the FISH-treated cells is positioned into the X-Y stage 201. The X-Y stage 201 is moved to an initial observation position found to contain a rare cell. A processing loop is executed repeatedly until either a predetermined number of the rare cells of a particular type have been measured. In the application for which the present embodiment is intended, identifying multiple targets of chromosomal DNA, the loop is executed until 20-100 nuclei have been processed. Data representing the measurement of the chromosomal areas within those nuclei may be collected in an ASCII file.

The filtering steps 12000 may operate on a pixel-by-pixel basis, as follows. In step 12001, a hole filling filter is applied to the image. This filter, available through the SIMPLE language, determines when dark holes have appeared within the lighter fluorescent chromosomes by searching for dark areas within light objects. Those areas are lightened up. The output of the hole filling filter is held in a temporary image file 12101, as well as being used as the input to the erosion filter, step 12003. Erosion filtering, also available through the SIMPLE language, replaces the center pixel of a small kernel with the darkest pixel in the kernel. The kernel used is 3×3 may be used. A separate operation, step 12005 is next performed, to grow the objects until they meet, but do not merge. This step also creates outlines, defining the edges of all the objects. A logical NOT operation, step 12007, causes the pixels within the outlines to become selected rather than the outlines. Finally, in step 12009, the result of step 12007 is logically ANDed with the stored temporary image file 12101. This causes only those pixels which are defined in both the temporary image file 12101 and the output of step 12007 to be retained.

If a combination of fluorescence detection procedures is used, more than two chromosomal areas may be detected per nucleus. Therefore, it is possible to recognize two chromosomal areas relative to chromosomes 21, another two relative to chromosome 18, one relative to chromosome X and one relative to chromosome Y, enabling the discovery of possible numerical aberrations detected by the enumeration of hybridization signals. The enumeration of the hybridization signals may be executed after completing the measurement of 20100 nuclei through an application program external to SIMPLE, compiled using CLIPPER (COMPUTER ASSOCIATES, CA). This program reads the measurement results ASCII file and classifies the chromosomal areas detected according to their RGB color combination. When two or more different fluorophors are used in combination, different combinations of ROB color values may be used to distinguish different targets, some targets of which may be labeled by more than one fluorophor. For example, targets may be stained with red and green fluorophors, but one target may receive fluorophors to emit 30% red and 70% green, another target may receive fluorophors to emit 70% red and 30% green, while a third target may receive fluorophors to emit only red. The three targets may be distinguished on the basis of their relative emissions. If the number of signals indicative of a chromosomal area corresponding to a specific chromosome, e. g., chromosome 21, is greater than two to an operator-selected statistically significant level, then a report is issued identifying an increased likelihood for trisomy 21 in the specific sample.

Although the present invention has been described in connection with the clinical detection of chromosomal abnormalities in a cell-containing sample, the image processing methods disclosed herein has other clinical applications. For example, the image processing steps described can be used to automate a urinalysis process. When the techniques of the present application are combined with those of application Ser. No. 08/132,804, filed Oct. 7, 1993, a wide variety of cell types can be visualized and analyzed, based on their morphology. Cell morphology can be observed for the purpose of diagnosing conditions for which cell morphology has been correlated to a physiological condition. Such conditions are known to those of skill in the art. See, e. g., Harrison, supra. Various cell characteristics and abnormalities may be detected based on these techniques. Finally, it should be noted that the particular source of the sample is not a limitation of the present invention, as the sample may be derived from a blood sample, a serum sample, a urine sample or a cell sample from the uterine cervix. The cell visualization and image analysis techniques described herein may be used for any condition detectable by analysis of individual cells, either by morphology or other characteristics of the isolated cells.

Antibodies specific for human fetal hemoglobin (Research Diagnostics Inc., NJ) and for embryonic epsilon hemoglobin chain (Immuno-Rx, GA) are commercially available and can be used as fluorescently labeled antibodies or a fluorescent signal can be generated by use of a fluorescently labeled secondary antibody. Fluorescent light can be produced by other types of stains or labels for rare cells, as known in the art. Fluorescent staining of the type required for this processing step is known in the art, and will not be discussed in further detail.

Computer and image processing technologies are constantly changing. Newer technologies which meet the needs of the above-described methods and apparatus, while not specifically described here, are clearly contemplated as within the invention. For example, certain conventional pixel and image file formats are mentioned above, but others may also be used. Image files may be compressed using JPEG or GIF techniques now known in the art or other techniques yet to be developed. Processing may be performed in an RGB color description space instead of the HLS space currently used. Other color spaces may also be used, as desired by the skilled artisan, particularly when detection of a sought-after characteristic is enhanced thereby.

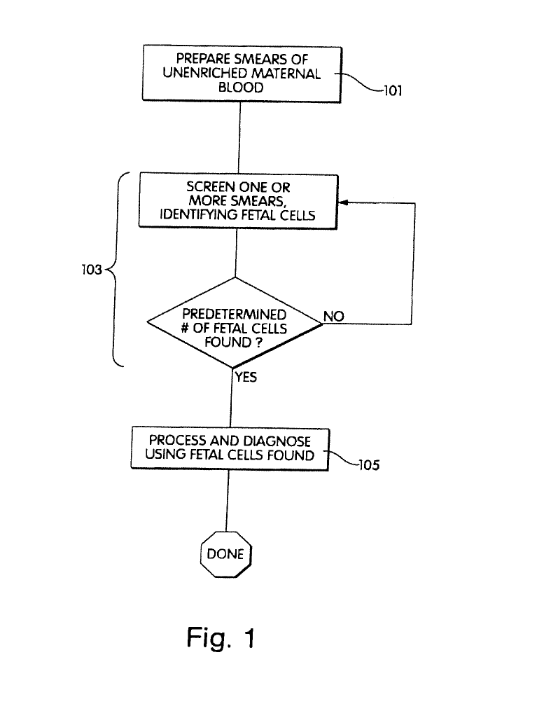

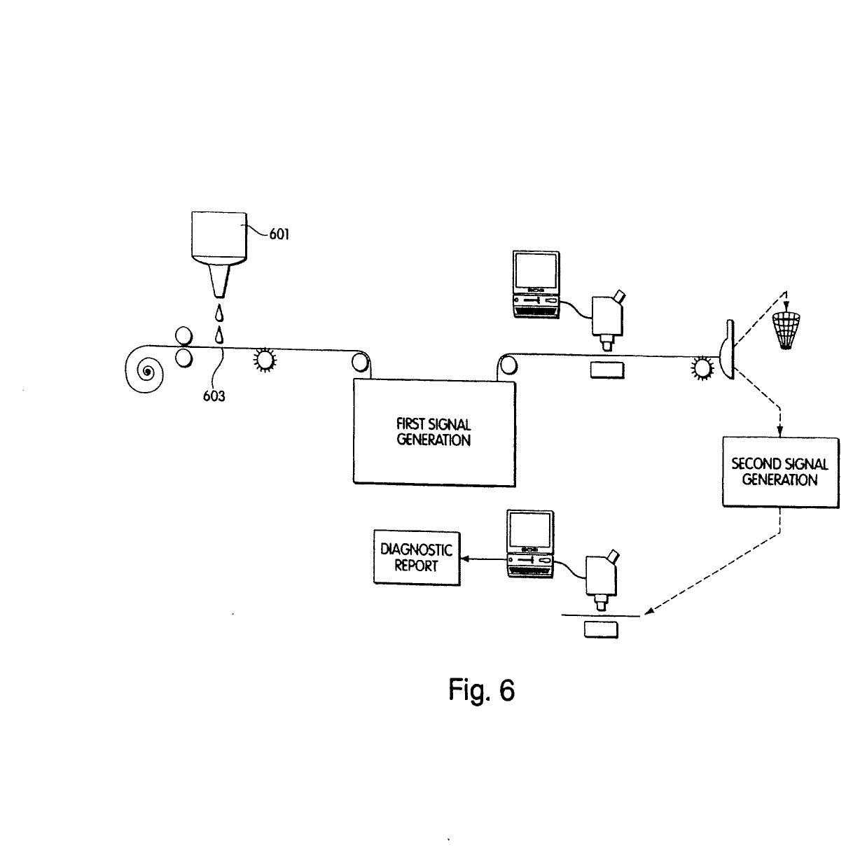

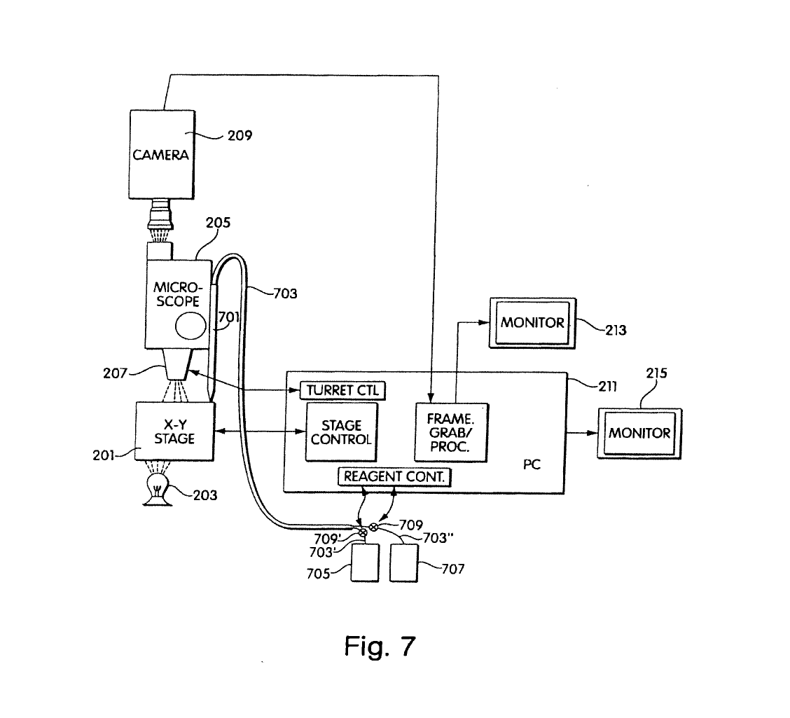

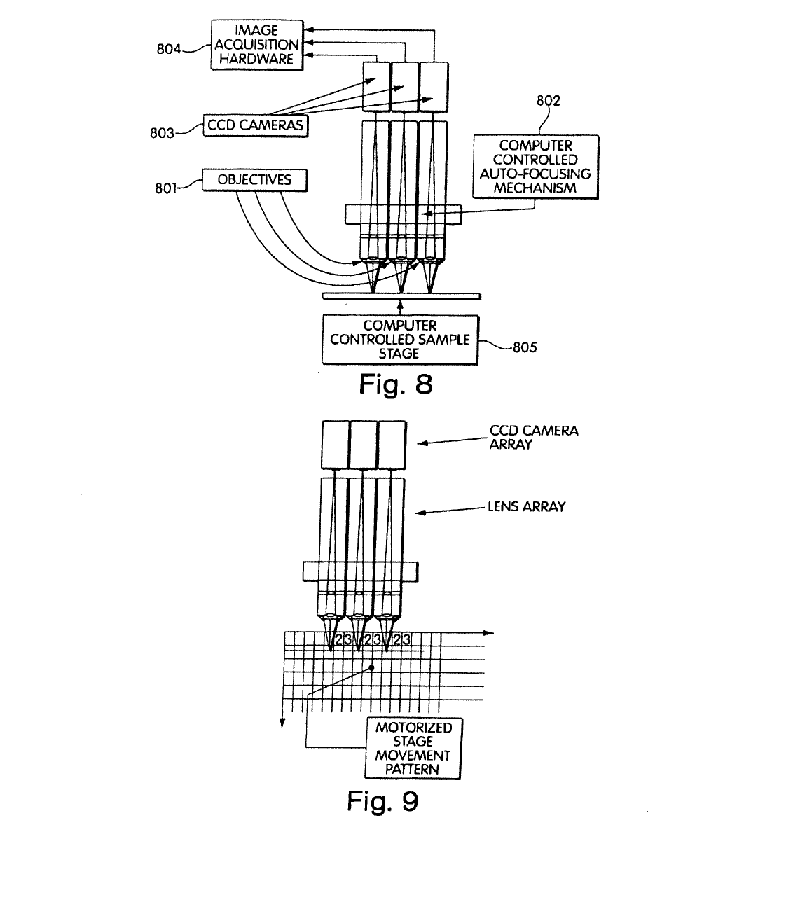

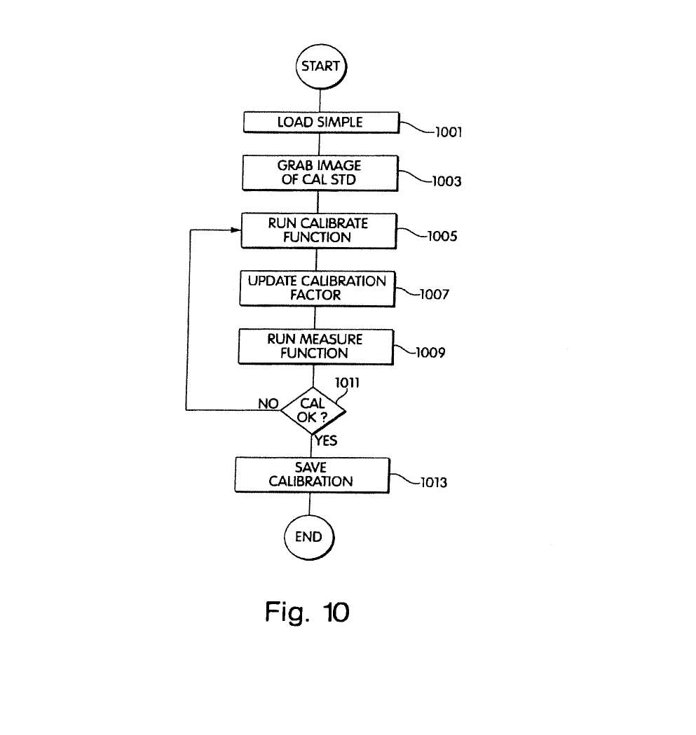

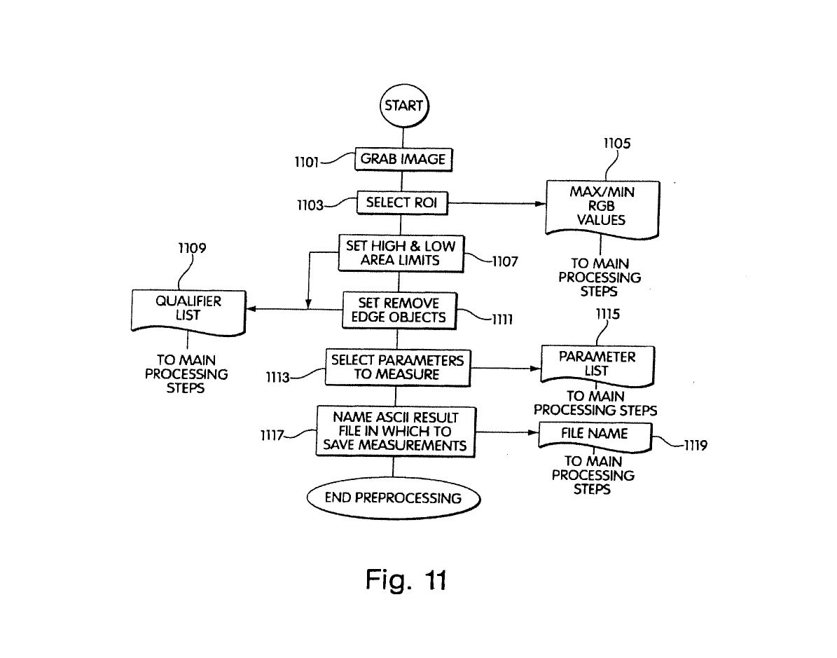

The present invention has now been described in connection with a number of particular embodiments thereof. Additional variations should now be evident to those skilled in the art, and are contemplated as falling within the scope of the invention, which is limited only by the claims appended hereto and equivalents thereof. It is further noted, that a detailed description of FIGS. 1, 6-10, 12B is provided in a number of earlier priority documents, e.g., in the priority patent application U.S. Ser. No. 10/130,559, filed on May 17, 2002 and issued as U.S. Pat. No. 7,346,200 on May 18, 2008 and in PCT application PCT/US99/27608 filed on Nov. 18, 1999 (Publication No. WO 01/37192, published May 25,2001) as incorporated here by reference.

In embodiments of the invention, there is illustrated an example for analysis of subcellular components of cells for the detection of for example, chromosomal abnormalities in prenatal and pre-implantation genetic diagnosis, or the sex chromosomes of embryonal or fetal cells.

A photomicrograph with multi-color representation of a combined immunostaining and FISH analysis of cells for the presence of fetal hemoglobin and the identification of X and Y chromosomes in the cells may be made. Fetal hemoglobin present in the sample may be shown, for example, by orange fluorescent signal detected from the cells and throughout the cytoplasm of the cell. X and Y chromosomes shown as green aqua red fluorescent dots, respectively, in the nucleus of the cells.

What is claimed is:

1. A method comprising:
receiving a cell sample that has been reacted with at least one antibody wherein each antibody binds to a specific cellular component and generates a unique antibody fluorescent signal;
wherein said cell sample has further been treated by in-situ hybridization using, at least, a first and a second nucleic acid probe, said first nucleic acid probe is constructed to hybridize with a first target nucleic acid sequence in said cell sample and generate a first unique fluorescent signal and said second nucleic acid probe is constructed to hybridize with a second target nucleic acid sequence in said cell sample and generate a second unique fluorescent signal;
capturing at least one image of said reacted and treated cell sample under conditions stimulating generation of said fluorescent signals;
detecting and counting said first unique fluorescent signals and said second unique signals and computing a ratio of the count said first unique fluorescent signals and said second unique signals; and
identifying and reporting the presence of a physiological condition based on said ratio.

2. The method in accordance with claim 1, wherein said first target nucleic acid sequence defines a dominant trait and said second target nucleic acid sequence defines a recessive trait.

3. The method in accordance with claim 1, further comprising the employment of said first and/or second nucleic acid probe chosen to hybridize with a break region between rearranged and non-rearranged nucleic acids.

4. The method in accordance with claim 1, wherein the detecting and counting step further comprises:
producing an image file of red, green and blue pixels representative of red, green and blue intensities at respective pixel locations within said at least one image;
receiving a manual selection of a plurality of pixels within the background;
determining color intensity value ranges corresponding to said background; and
identifying as background those areas of said at least one image having color intensity values within said color intensity value ranges corresponding to said background.
